(19) United States
(12) Patent Application Publication
(10) Pub. No.: US 2024/0341987 A1
(43) Pub. Date: Oct. 17, 2024

(54) AIR RELEASE QUANTIFICATION AND BACK-BLEED FLUSHING TECHNIQUES AND FEATURES FOR ENDOPROSTHESIS DELIVERY SYSTEM

(71) Applicant: W. L. Gore & Associates, Inc., Newark, DE (US)

(72) Inventors: Thomas E. Kariniemi, New Castle, CO (US); Paul Morgan, Gilbert, AZ (US); Martin J. Sector, Phoenix, AZ (US); Maya C. Uddin, Phoenix, AZ (US); Wenqi Yang, Phoenix, AZ (US)

(21) Appl. No.: 18/637,257

(22) Filed: Apr. 16, 2024

Related U.S. Application Data
(60) Provisional application No. 63/459,849, filed on Apr. 17, 2023.

(51) Int. Cl. A61F 2/966

(52) U.S. Cl. CPC A61F 2/966

(57) ABSTRACT

Techniques and features for reduction of air potentially released during endoluminal device (e.g., thoracic aortic stent-graft device) deployment. Also addressed are methods for quantifying efficacy of pre-treatment techniques and features for reducing said air. Such air reduction can help reduce risk of air embolization during device (e.g., implant, such as stent-graft) deployment which could potentially decrease the risk of negative effects from such embolization.

### CROSS-REFERENCE TO RELATED APPLICATION

This application claims the benefit of Provisional Application No. 63/459,849, titled “Air Release Quantification and Back-Bleed Flushing Techniques and Features for Endoprosthesis Delivery System,” filed Apr. 17, 2023, which is incorporated herein by reference in its entirety for all purposes.

### BACKGROUND

Endoluminal devices are commonly delivered into the body of a patient (e.g., into the patient's vasculature) using an introducer system. Introducer systems typically include valves or similar features to stop backflow of body fluids (e.g., blood) through to the introducer while permitting the endoluminal device to pass through the introducer and into the patient's body. In various instances, a clinician or other user of an endoluminal device may also wish to perform one or more treatments on the endoluminal device.

Particularly advantageous introducer systems include those sold by W. L. Gore & Associates, Inc. under the tradename GORE® DrySeal Flex Introducer Sheath. GORE® DrySeal Flex Introducer Sheaths are intended to be inserted in the vasculature to provide conduits for insertion of endovascular devices while minimizing blood loss associated with such insertions. The GORE® systems include an introducer sheath with the GORE® DrySeal Valve attached, a twist style locking dilator, and a syringe. The introducer sheath is a composite tube which consists of a flat stainless-steel wire reinforced hydrophilic coated Pebax® outer tube and PTFE liner with a tapered leading tip. The introducer sheath is attached to the GORE® DrySeal Valve. The GORE® DrySeal Valve includes an outer silicone tube and an inner film tube. The region between the silicone tube and film tube can be pressurized by injecting saline into the region using a syringe. Additional examples of similar systems can be found in U.S. Pat. No. 10,155,104, “Valve Assembly for Medical Procedures,” filed by W. L. Gore & Associates, Inc.

Some methods of endoluminal device pre-treatment before device deployment and/or delivery to a target site in the body include inserting an endoprosthesis delivery catheter for an endoprosthesis device (GORE® TAG® Conformable Thoracic Stent Graft) over a guidewire into the valve of an introducer sheath (GORE® DrySeal Sheath), stopping when only the trailing end of the constrained endoprosthesis device is visible, and allowing blood to flow back through the constrained endoprosthesis device.

### SUMMARY

Various techniques and features addressed herein relate to reduction of air potentially released during endoluminal device (e.g., thoracic aortic stent-graft device) deployment. Also addressed are methods for quantifying efficacy of pre-treatment techniques and features for reducing said air. Such air reduction can help reduce risk of air embolization during device (e.g., implant, such as stent-graft) deployment which could potentially decrease the risk of negative effects from such embolization.

According to one example (“Example 1”), an endoluminal device delivery system includes an endoluminal device including, an implant having a first end and a second end, the implant being configured in a collapsed, delivery configuration, the implant having one or more void spaces defined by the collapsed implant and filled with air; a delivery catheter including a body to which to the endoluminal device is mounted; and a treatment system including, a delivery sheath, and a valve coupled to the delivery sheath, the valve being actuatable between a sealed and an unsealed configuration, the implant projecting proximally from the valve a projection distance of approximately 1 cm, the treatment system defining a blood pathway between the delivery sheath and the body of the catheter for blood to pass from the first end of the implant to the second end of the implant and into the one or more void spaces defined by the collapsed implant, the valve actuatable from an unsealed state in which blood bypasses the blood pathway to a sealed state in which blood is forced along the blood pathway.

According to one example (“Example 2”), further to Example 1, wherein the implant projects proximally from the valve from 0.5 cm to 1.5 cm.

According to one example (“Example 3”), further to Examples 1 or 2, wherein the implant includes an implant visual insertion marker corresponding to the projection distance.

According to one example (“Example 4”), further to Examples 1 to 3, wherein the endoluminal device further includes a retention sleeve maintaining the implant in the collapsed, delivery configuration.

According to one example (“Example 5”), further to Example 4, wherein the retention sleeve includes a sleeve visual insertion marker corresponding to the projection distance.

According to one example (“Example 6”), an endoluminal device delivery system includes an endoluminal device including, an implant having a first end and a second end, the implant being configured in a collapsed, delivery configuration, the implant having one or more void spaces defined by the collapsed implant and filled with air; a delivery catheter including a body to which to the endoluminal device is mounted; and a treatment system including, a delivery sheath, and a seal between the delivery catheter and the delivery sheath at a location proximal the implant corresponding to a projection distance between the seal and the implant, the delivery sheath having a distal end configured to be exposed to blood pressure and the treatment system having one or more vents configured to vent to ambient air pressure, the one or more vents having an opening located distal to the seal and proximal the implant such that the treatment system defines a blood pathway between the delivery sheath and the body of the catheter for blood to pass from the first end of the implant to the second end of the implant and out of the one or more vents, the blood being directed into the one or more void spaces defined by the collapsed implant.

According to one example (“Example 7”), further to Example 6, wherein the one or more vents has an exit located distal to the seal.

According to one example (“Example 8”), further to Example 7, wherein the one or more vents are formed in the delivery sheath.

According to one example (“Example 9”), further to Example 6, wherein the one or more vents has an exit located proximal to the seal.

According to one example (“Example 10”), further to Example 9, wherein the exit is located at the proximal end of the treatment system.

According to one example (“Example 11”), further to Examples 9 or 10, wherein the one or more vents are formed in the delivery catheter.

According to one example (“Example 12”), further to Examples 9 to 11, wherein the one or more vents are formed as a vent tube, lumen, or snorkel, providing a fluid conduit from distal to seal to a proximal opening located toward the proximal end of the treatment system.

According to one example (“Example 13”), further to Example 6 to 12, wherein the implant includes an implant visual insertion marker corresponding to the projection distance.

According to one example (“Example 14”), further to Example 13, wherein the implant visual insertion marker includes a radiographic material.

According to one example (“Example 15”), further to Examples 6 to 12, wherein the delivery catheter includes a delivery catheter visual insertion marker corresponding to the projection distance.

According to one example (“Example 16”), an endoluminal device delivery system having a pre-selected projection distance of an endoluminal device from a delivery sheath of the system, the system includes an endoluminal device including, an implant having an outer surface and being configured to transition from a diametrically compacted, delivery configuration to a diametrically expanded, deployed configuration, the implant having a proximal end, a distal end opposite to the proximal end, and length between the proximal and distal ends, and a retention sleeve constraining the implant in the diametrically compacted, delivery configuration, wherein at least one of the implant and the retention sleeve includes a visual insertion marking on an outer surface thereof; and a treatment system including a delivery sheath configured to introduce the endoluminal device into a body of a patient, wherein the visual insertion marking corresponds to the pre-selected projection distance of the endoluminal device from the delivery sheath.

According to one example (“Example 17”), further to Example 16 wherein the visual insertion marking is visible with the unaided eye.

According to one example (“Example 18”), further to Examples 16 or 17, wherein the visual insertion marking aligns with a proximal end of the treatment system at the pre-selected projection distance.

According to one example (“Example 19”), an endoluminal device delivery system having a pre-selected projection distance of an endoluminal device relative to a seal of a delivery sheath of the system, the system includes an endoluminal device including, an implant having a first end and a second end, the implant being configured in a collapsed, delivery configuration, the implant having one or more void spaces defined by the collapsed implant and filled with air; a delivery catheter including a body to which to the implant is mounted; and a treatment system including, a delivery sheath, and a seal between the delivery catheter and the delivery sheath at a location proximal the implant corresponding to a projection distance between the seal and the implant, wherein at least one of the implant and the delivery catheter includes a visual insertion marking on an outer surface thereof corresponds to the pre-selected projection distance of the endoluminal device from the delivery sheath.

According to one example (“Example 20”), further to Example 19, wherein the visual insertion marking is visible with the unaided eye.

According to one example (“Example 21”), further to Examples 19 or 20, wherein the visual insertion marking aligns with a proximal end of the treatment system at the pre-selected projection distance.

According to one example (“Example 22”), a method of pre-treating an endoluminal device by back-bleeding, the method includes positioning a treatment system in a body lumen of a patient such that a distal portion of the treatment system is exposed to blood at a blood pressure; inserting an endoluminal device including an implant in a diametrically compacted, collapsed state and a delivery catheter into the treatment system at a pre-selected projection distance, the treatment system including a delivery sheath and a seal between the endoluminal device and the treatment system; permitting blood to flow along a blood pathway defined between the distal portion of the treatment system and a proximal portion of the treatment system, the blood pathway extending through a first end of the implant to the second end of the implant and into one or more void spaces defined by the implant such that a volume of entrained air in the one or more void spaces is reduced.

According to one example (“Example 22”), further to Example 21, wherein the volume of the entrained air is reduced to 10 μL or less.

According to one example (“Example 23”), further to Example 21, wherein the volume of the entrained air is reduced to 5 μL or less.

According to one example (“Example 24”), further to Example 21, wherein the volume of the entrained air is reduced to 2 μL or less.

According to one example (“Example 25”), further to Examples 21 to 24, wherein at least one of the implant, the delivery catheter, and a retention sleeve of the endoluminal device includes a visual insertion marking on an outer surface thereof that corresponds to the pre-selected projection distance, and further wherein the method includes inserting the endoluminal device into the treatment system to the pre-selected projection distance using the visual insertion marking.

According to one example (“Example 26”), further to Example 25, wherein the method includes using the visual insertion marking with the unaided eye.

According to one example (“Example 27”), further to Examples 21 to 26, wherein permitting blood to flow along the blood pathway includes observing 2 to 3 drips of blood from the blood pathway.

### BRIEF DESCRIPTION OF THE DRAWINGS

FIG. 1 shows an endoluminal device delivery system 10, according to some embodiments.

FIG. 2 shows a longitudinal section of a portion of the treatment system proximate the valve, according to some embodiments.

FIG. 3 is an isometric view of the same portion of the treatment system as FIG. 2, but in a disassembled state, according to some embodiments.

FIG. 4 is an end view of the treatment system showing the valve in a pressurized, and sealed state according to some embodiments.

FIGS. 5 and 6 show an endoluminal device in the form of a transcatheter delivery system including a catheter and an implantable device maintained at a compacted, delivery diameter or state by the delivery catheter, according to some embodiments.

FIG. 7 shows an endoluminal device in the form of a transcatheter delivery system including a catheter and an implantable device maintained at a compacted, delivery diameter or state by the delivery catheter, according to some embodiments.

FIG. 8 shows an endoluminal device in the form of a transcatheter delivery system including a catheter and an implantable device maintained at a compacted, delivery diameter or state by the delivery catheter, according to some embodiments.

FIG. 9 is a close-up, schematic, sectional, and partial view of a portion of the treatment system of FIG. 1 with the endoluminal device in a treatment position, according to some embodiment methods of pre-treating the endoluminal device using a back-bleed method.

FIGS. 10 to 12 illustrate an example treatment sequence for an endoluminal device using the treatment system of FIG. 1, according to some embodiments.

FIG. 13 is a schematic, sectional view of another endoluminal device delivery system, according to some embodiments.

FIG. 14 is a schematic, sectional view of yet another endoluminal device delivery system, according to some embodiments.

FIGS. 15 to 19 show various features of an Air Release Test (ART), according to some embodiments.

FIGS. 20 to 25 show data corresponding to various example systems, according to some embodiments.

The accompanying drawings are included to provide a further understanding of the disclosure and are incorporated in and constitute a part of this specification, illustrate embodiments, and together with the description serve to explain the principles of the disclosure. Persons skilled in the art will readily appreciate that various aspects of the present disclosure can be realized by any number of methods and apparatuses configured to perform the intended functions. It should also be noted that the accompanying drawing figures referred to herein are not necessarily drawn to scale, but may be exaggerated to illustrate various aspects of the present disclosure, and in that regard, the drawing figures should not be construed as limiting.

### DETAILED DESCRIPTION

### Definitions and Terminology

This disclosure is not meant to be read in a restrictive manner. For example, the terminology used in the application should be read broadly in the context of the meaning those in the field would attribute such terminology.

With respect to terminology of inexactitude, the terms “about” and “approximately” may be used, interchangeably, to refer to a measurement that includes the stated measurement and that also includes any measurements that are reasonably close to the stated measurement. Measurements that are reasonably close to the stated measurement deviate from the stated measurement by a reasonably small amount as understood and readily ascertained by individuals having ordinary skill in the relevant arts. Such deviations may be attributable to measurement error, differences in measurement and/or manufacturing equipment calibration, human error in reading and/or setting measurements, minor adjustments made to optimize performance and/or structural parameters in view of differences in measurements associated with other components, particular implementation scenarios, imprecise adjustment and/or manipulation of objects by a person or machine, and/or the like, for example. In the event it is determined that individuals having ordinary skill in the relevant arts would not readily ascertain values for such reasonably small differences, the terms “about” and “approximately” can be understood to mean plus or minus 10% of the stated value.

### DESCRIPTION OF VARIOUS EMBODIMENTS

Various techniques and features addressed herein relate to reduction of air potentially released during endoluminal device (thoracic aortic stent-graft device). Also addressed are methods for quantifying efficacy of pre-treatment techniques and features for reducing said air. Such air reduction can help reduce risk of air embolization during stent graft deployment which could potentially decrease the risk of negative effects from such embolization.

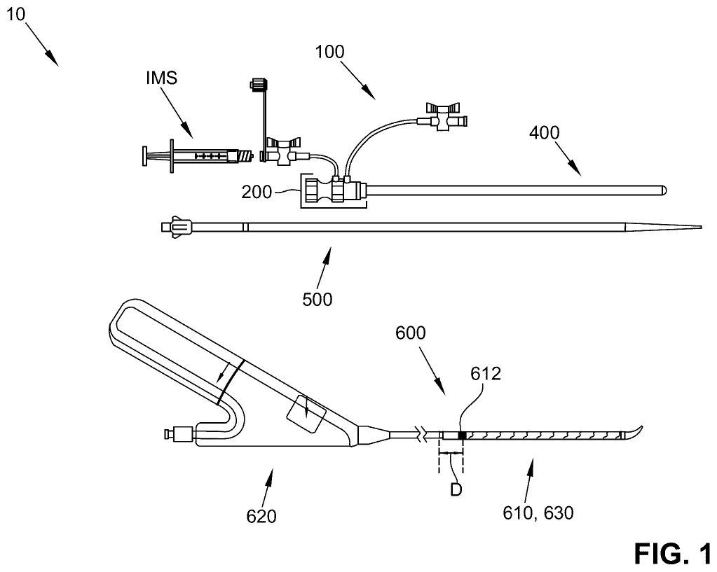

FIG. 1 shows an endoluminal device delivery system 10, according to some embodiments. The system 10 includes a treatment system 100 (optionally described as an introducer system) including a valve 200, a delivery sheath 400 and an optional dilator 500. The system 10 also includes an endoluminal device 600 (e.g., in the form of a transcatheter delivery system including a catheter and an implantable device maintained at a compacted, delivery diameter or state by the delivery catheter). The endoluminal deice 600 includes an implant 610, or implantable device 610 carried by a delivery catheter 620. The endoluminal device 600 also includes a retention sleeve 630 for constraining the implant 610.

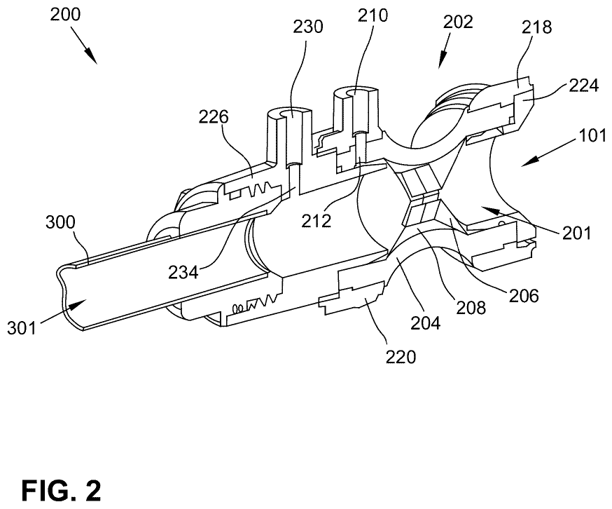

FIG. 2 shows a longitudinal section of a portion of the treatment system 100 proximate the valve 200. In general terms, the treatment system 100 includes an inner lumen 101 that extends continuously through the full length of the treatment system 100, including through the valve and the delivery sheath 400 configured to facilitate introduction of an endoluminal device into a body lumen.

The treatment system 100 may be used to introduce an endoluminal device into a body of a patient (not shown) by passing an endoluminal device through the inner lumen 101 from a location external to a body of a patient to a location inside the patient. The treatment system 100 may be utilized with a variety of endoluminal devices, such as those illustrated in FIGS. 1 and 6 to 9. In various examples, the treatment system 100 is used to treat the endoluminal device (e.g., flush air from a portion of the endoluminal device) with a back-bleed method (e.g., using blood at arterial pressures to flush, or de-aerate one or more portions, such as the implant, of the endoluminal device 600).

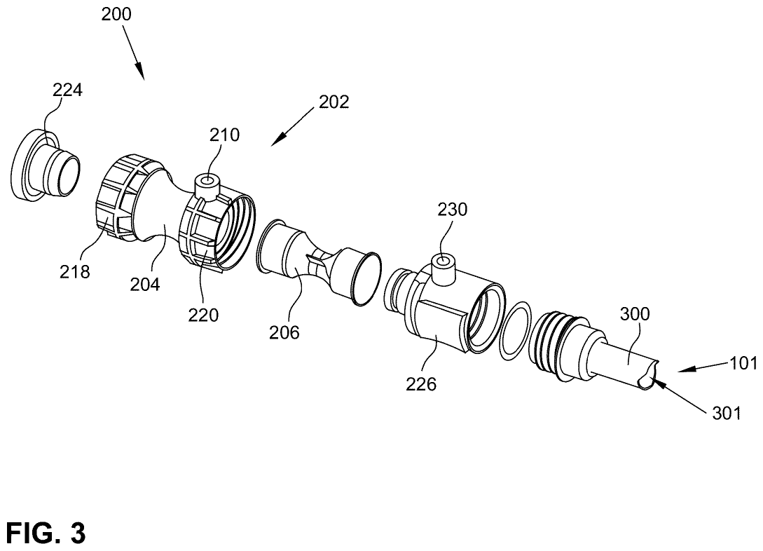

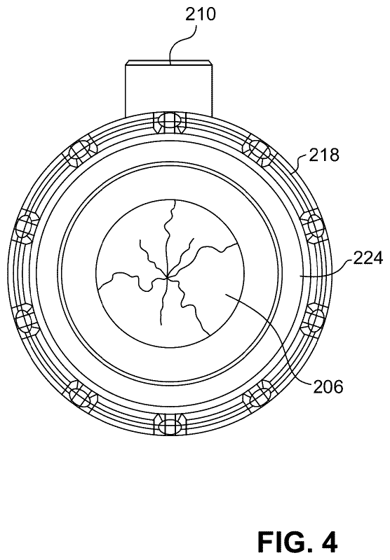

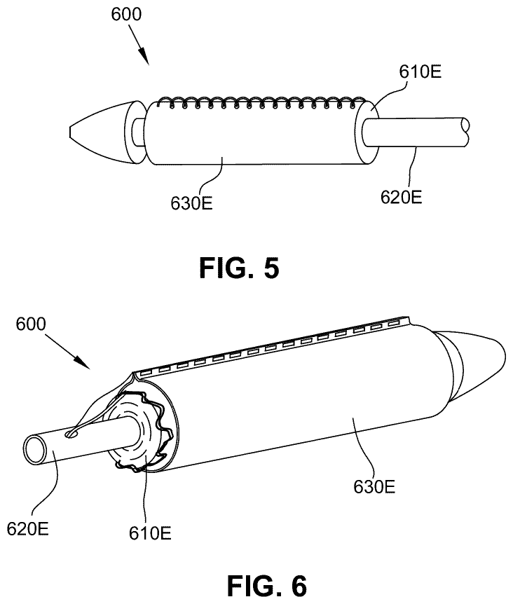

FIG. 2 is an enlarged, partial cut-away view of a proximal portion of the treatment system 100 showing various features of the proximal valve 200 in an assembled state. FIG. 3 is an isometric view of the same portion of the treatment system 100 as FIG. 2, but in a disassembled state. FIG. 4 is an end view of the treatment system 100 showing the valve 200 in a pressurized, and sealed state according to some examples.

The valve 200 is generally configured to receive an endoluminal device (e.g., dilator, endovascular delivery systems, balloon catheters, percutaneous delivery systems, and the like) and provide a fluid seal around the outer surface of the endoluminal device to prevent unwanted backflow (e.g., of blood and/or a treatment medium) around the endoluminal device and back through the inner lumen 101 of the treatment system 100. Additionally, the valve 200 is configured to fully close, or close off and seal with itself to a closed, or sealed state when no endoluminal device is present (e.g., as shown in FIG. 4). Again, this prevents unwanted backflow (e.g., of blood and/or a treatment medium) through the proximal valve 200. Suitable examples of designs, materials, and methods of making the proximal valve 200 and delivery sheath 400 may be found in U.S. Pat. No. 10,155,104, entitled “Valve Assembly for Medical Procedures,” although a variety of designs, materials and methods of making such valves are contemplated. As discussed, the valve 200 and delivery sheath 400 may correspond to the GORE® DrySeal Flex Introducer Sheath available from W. L. Gore & Associates, Inc.

As can be seen in FIG. 2, the valve 200 has an inner lumen 201 that extends the length of the valve 200. As previously referenced, the inner lumen 201 of the valve 200 forms a portion of the inner lumen 101 of the treatment system 100. The valve 200 includes a seal mechanism 202 that is actuatable between a sealed state and an unsealed state to open and close, or dilate, a portion of the inner lumen 201. The seal mechanism 202 includes an outer tube 204, an inner tube 206, a pressurizable space 208 (FIG. 2) formed between an inner surface of the outer tube 204 and an outer surface of the inner tube 206, and a fill port 210. Though apparent from the figures, it is noted that the pressurizable space 208 is generally sealed off apart from the access provided by the fill port 210 according to various examples. As illustrated, the seal mechanism 202 also includes a rear ring 218 and a front ring 220 secured in an opposing manner toward either end of the outer tube 204. The rings generally assist with supporting, sealing, and coupling the seal mechanism 202 with a remainder of the valve.

The valve 200 also includes a rear fitting 224 attached to the rear ring 218 and a front fitting 226 attached to the front ring 220 (e.g., via complementary threads, adhesives, snap fits, fasteners and/or other mechanisms). The rear and front fittings 224, 226 can help to secure the various portions of the valve together in a sealed manner and may also provide a mechanism or manner for securing the valve to other components of the treatment system 100, such as the delivery sheath 300.

In addition to the fill port 210, the valve 200 also has a flush port 230 (also described as a flush port) in fluid communication with the inner lumen 201 of the valve at a location distal to the seal mechanism 202. As will be subsequently described, the flush port 230 may be utilized to deliver a treatment medium into the treatment chamber 300.

In some embodiments, the outer tube 204 has an hourglass shape in a relaxed state, although right cylinder and other shapes are contemplated. The outer tube 204 may have elastic properties (e.g., being formed of an elastomeric material) and distend (physically expand) upon pressurization of the pressurizable space 208 to deflect radially outward from the hourglass shape to a more cylindrical state and potentially a more bulbous, outwardly convex shape. In some examples, the outer tube 204 is formed of a silicone material (e.g., using insertion molding techniques), although a variety of materials including any of a variety of elastomeric materials or materials having elastic properties are contemplated. For example, the outer tube 204 may be constructed of any elastomer, latex or polycarbonate with desirable mechanical and biocompatible properties.

The expansion characteristic of the outer tube 204 can provide a visual indicator that the pressurizable space 208 has been positively pressurized, and thus the valve has been closed. In some examples, when the seal mechanism 202 is positively pressurized and closed, the hourglass shape of outer tube 204 becomes distended to indicate a desirable positive pressure in the pressurizable space 208 (e.g., one that will sufficiently prevent backflow through the seal mechanism 202).

The inner tube 206 may be constructed of any thin, strong, drape-able material such as ePTFE, fabrics, silk, or Kevlar® brand fiber, for example. Such materials may be used as a single layer construct or a multi-layer construct as appropriate. As shown, the inner tube 206 may have an hourglass shape in a relaxed state. The shape of the inner tube 206, can be varied as desired, including wall thickness, length, width, diameter, and other features.

In use, the inner tube 206 is thin and conformable and as such, once the pressurizable space 208 is positively pressurized, the inner tube 206 is deflected inwardly and drapes, or closely conforms, to the outer perimeter of an endoluminal device received through the valve to form a seal. When no endoluminal device is present, the inner tube 206 deflects inwardly such that the inner surface of the inner tube 206 engages itself to form a seal.

As shown, the fill port 210 includes a coupling feature 211 associated with the front ring 220 and a passage 212 formed through the outer tube 204 into the pressurizable space 208. The coupling feature 211 is optionally configured to be attached to a syringe (e.g., the coupling feature 211 can be configured as a valved luer fitting). Regardless of the particular pathway, the fill port 210 provides a means for pressurizing (or depressurizing) the pressurizable space 208. In particular, the fill port 210 is in fluid communication with the pressurizable space 208.

The fill port 210 can be configured to be coupled to any of a variety of positive or negative pressure sources (fluid or gas), including a syringe (not shown). For reference, the pressurizable space 208 may be filled with any suitable material or materials. For example, although saline solution may be preferred in certain applications, the pressurizable space 208 may be positively pressurized with one or more of: air, silicone, water, saline solution, low volatility biocompatible liquids, glycerin, propylene glycol, polyethylene glycol, compressible foam, elastomeric spheres, crosslinked silicone gels, and combinations thereof.

Regardless, a pressure source can be used to deliver a suitable material (e.g., saline solution) into the pressurizable space 208 (to positively pressurize) or to remove material from the pressurizable space 208 (to negatively pressurize, or depressurize), respectively, causing closing or opening, respectively, of the seal mechanism 202. In particular, according to various embodiments, upon positive pressurization of the pressurizable space 208 using the fill port 210, the inner tube 206 collapses inwardly (e.g., against itself or around a device received through the inner tube 206) to form a seal. FIG. 4 shows an end view of the treatment system 100 with the inner tube 206 of the valve collapsed and engaged with itself under positively pressurized conditions.

The front fitting 226 is secured to the front ring 220 as well as a proximal portion of the treatment chamber 300 (e.g., via complementary threading as shown in FIG. 2), and thus assists with fluidly coupling the valve to the treatment chamber 300. The front fitting 226 or portions thereof can be formed using a clear material (e.g., transparent or translucent polymer) which can permit visual confirmation by a user of the treatment system 100 that a device (or portion thereof) being inserted through the treatment system 100 has passed through the valve, and in particular beyond the seal mechanism 202.

As shown, the flush port 230 communicates with the inner lumen 201 at a location distal to the seal mechanism 202. The flush port 230 includes a coupling feature 232 (e.g., a valved luer fitting for sealing and unsealing the flush port 230) and a passage 234 through the front fitting 226 into the inner lumen 201 of the valve (e.g., at a location distal to the seal mechanism 202 as shown). Regardless of the particular pathway, the flush port 230 provides a means for conveying a treatment medium into and/or out from the inner lumen 201 of the valve (and into or out from a proximal portion of the treatment chamber 300) as subsequently described.

As shown in FIG. 1, the delivery sheath 400 is coupled to the front fitting 226 of the valve 200 (e.g., via complementary threading on the two components). The delivery sheath 400 is substantially tubular and has an inner lumen (not shown) that forms a portion of the inner lumen 101 of the treatment system 100. The delivery sheath 400 may be formed of a variety of materials, but in some examples is formed of fluorinated ethylene propylene (FEP), high-density polyethylene, and/or any other suitable material. The delivery sheath 400 may be configured with an outer dimeter ranging between a variety of sizes, but in some examples is sized from 12 Fr to 26 Fr. The delivery sheath 400 may have any of a variety of lengths as desired and may be configured for insertion into a body lumen (e.g., vasculature) of a patient to assist with the introduction of an endoluminal device into the patient (not shown).

In some examples, portions of the treatment system 100 are transparent (e.g., portions of the valve 200 and/or delivery sheath 400), such that a user may better, or more easily visually confirm proper positioning of the endoluminal device 600 within the treatment system 100.

The optional dilator 500 can be used in combination with the treatment system 100 to access one or more body lumens of a patient. In use, the dilator 500 is received through the inner lumen 101 of the treatment system 100 and is utilized in association with the treatment system 100 to gain access to a patient's body (e.g., vasculature, airways, biliary tract, gastrointestinal tract, cardiac spaces, or others). The various valves of the treatment system 100 assist in preventing back bleeding through the treatment system 100 during a dilation procedure using the dilator 500. As shown, the dilator 500 includes a dilator tip 502, a dilator body 504 and a hub end 506. The hub end 506 is configured to connect with a proximal portion of the treatment system 100 and can also help prevent backout of the dilator 500 during insertion of the dilator 500 into the patient's body.

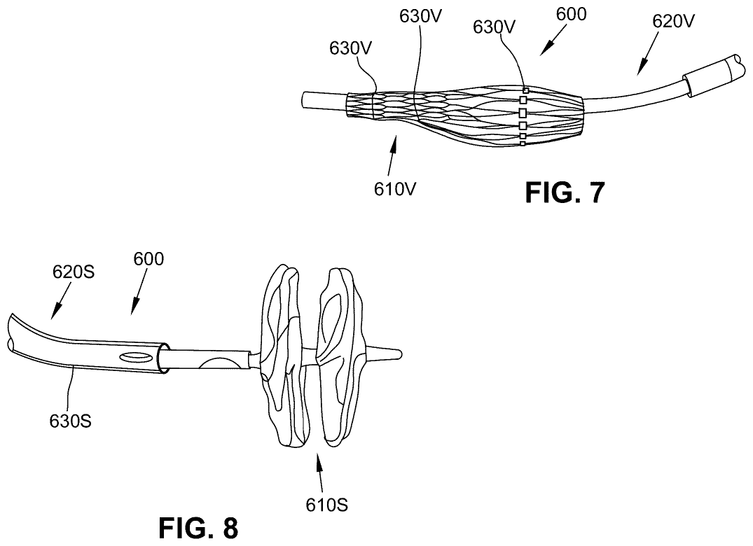

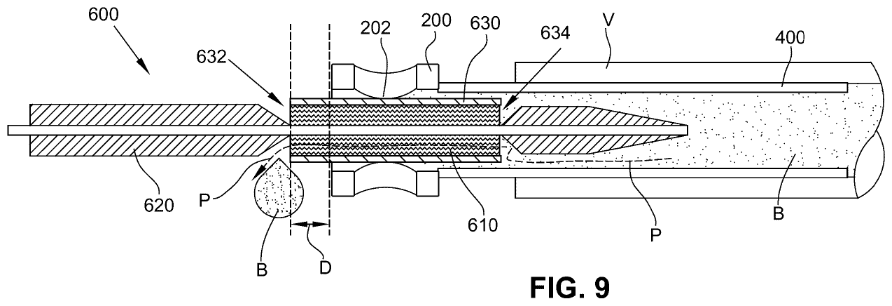

FIGS. 6 and 7 show an endoluminal device 600 in the form of a transcatheter delivery system including a catheter and an implantable device maintained at a compacted, delivery diameter or state by the delivery catheter. The implantable device defines a flush portion, or a part of the endoluminal device 600 for which a treatment is to be performed upon. In particular, the endoluminal device 600 in FIGS. 8 and 9 includes an endoprosthesis 610E (e.g., stent graft) maintained in a diametrically compacted state by a delivery catheter 620E. As shown, the delivery catheter 620E employs a retention sleeve constraining delivery system, such as that described in U.S. Pat. No. 9,592,143, entitled “Sleeves for Expandable Medical Devices.” The endoprosthesis 610E can be transferred between a fully compacted, delivery diameter or state (e.g., as shown in FIGS. 6 and 7) and a fully expanded, deployed diameter using the delivery catheter 620E, which releases the retention sleeve 630E to deploy the endoprosthesis 610E carried by the delivery catheter 620E.

FIG. 7 shows an endoluminal device 600 in the form of a transcatheter delivery system including a catheter and an implantable device maintained at a compacted, delivery diameter or state by the delivery catheter. The implantable device defines a flush portion, or a part of the endoluminal device 600 for which a treatment is to be performed upon. In particular, the endoluminal device 600 in FIG. 7 includes a prosthetic valve 610V (e.g., a prosthetic heart valve) maintained in a diametrically compacted state by a delivery catheter 620V. As shown, the delivery catheter 620V employs a fiber constraining delivery system, such as that described in U.S. application Ser. No. 16/129,657, entitled “Transcatheter Deployment Systems and Associated Methods,” filed Sep. 12, 2018. The prosthetic valve 610V can be transferred between a fully compacted, delivery diameter or state, a partially compacted treatment diameter (e.g., as shown in FIG. 7), and a fully expanded, deployed diameter using the delivery catheter 620V, which tightens and loosens constraints 630V releasably coupled to the prosthetic valve 610V.

FIG. 8 shows an endoluminal device 600 in the form of a transcatheter delivery system including a catheter and an implantable device maintained at a compacted, delivery diameter or state by the delivery catheter. The implantable device is included in the flush portion, or part of the endoluminal device 600 for which a treatment is to be performed upon. In particular, the endoluminal device 600 in FIG. 8 includes a septal occluder 610S (e.g., atrial septal occluder) maintained in a diametrically compacted state by a delivery catheter 620S. As shown, the delivery catheter 620S employs a sheath, or tube constraining delivery system, such as that described in U.S. Pat. No. 8,956,389, entitled “Sealing Device and Delivery System.” The septal occluder 610S can be transferred between a fully compacted, delivery diameter or state, a partially compacted treatment diameter, and a fully expanded, deployed diameter (e.g., as shown in FIG. 8) using the delivery catheter 620S, which extends and retracts the septal occluder 610S from sheath, or tube 630S associated with the delivery catheter 620S.

In some examples, once the implantable device 610 has been treated as desired (e.g., flushed of air, wetted out with blood, or otherwise treated), the implantable device 610 may then be tracked to a desired location within the body by advancing the implantable device 610 through and out from the delivery sheath 400 of the treatment system 100 (e.g., by tracking the implantable device 610 over a guidewire).

FIG. 9 is a close-up, schematic, sectional, and partial view of a portion of the treatment system 100 of FIG. 1 with the endoluminal device 600 in a treatment position, according to some methods of pre-treating the endoluminal device 600 using a back-bleed method. The endoluminal device 600 may be, for example, a GORE® TAG® Thoracic Branch Endoprosthesis Aortic Component (“TBE Aortic Component”), GORE® TAG® Thoracic Branch Endoprosthesis Side Branch (SB) Component (“TBE Side Branch”), or GORE® TAG® Conformable Thoracic Stent Graft with ACTIVE CONTROL System (“CMDS”), available from W. L. Gore & Associates, Inc., headquartered in Newark, DE.

The 600 includes the implant 610 (e.g., stent graft) maintained in a diametrically compacted state by the retention sleeve 630 that is releasable using the delivery catheter 620 to which the implant 610 is releasably coupled. As shown, the delivery sheath 400 has been inserted into, and received within, a blood vessel V, such as an arterial vessel. The endoluminal device 600, and in particular the implant 610 and the retention sleeve 630 have been translated partially into the delivery sheath 400, extending on one side of the valve 200, with the implantable device 610 and the retention sleeve 630 partially located in the valve 200 and another portion projecting proximally from the valve 200 a desired amount. In particular, the implantable device crosses and extends proximally back from the seal mechanism 202. In examples where portions of the treatment system 100 are transparent (e.g., portions of the valve 200 and/or delivery sheath 400), a user may visually confirm proper positioning of the implant 610.

The valve 200 is pressurized and closed to form a seal around the endoluminal device 600, and in particular the portion of the implant 610 and the retention sleeve 630 in the seal mechanism 202. As shown, the retention sleeve 630 is open at a proximal end 632 and a distal end 634 thereof, with a blood pathway P being defined through the delivery sheath, into the open distal end 632, the implant 610 (e.g., through the diametrically compressed body formed by the implant 610) and out from the proximal end 632. In particular, the pressurized blood (e.g., at mean arterial pressure) is prevent from passing around the retention sleeve 630 and, due to blood pressure, passes along the blood pathway P, eventually “back-bleeding” through the implant and existing the implant 610 (e.g., externally to the body of the patient). The flow of blood from the implant 610 may be relatively slow (drip-by-drip). In particular, the pressure differential between the interior blood pressure (e.g., mean arterial pressure) and ambient external pressure at the back end of the valve 200 drives blood along the blood pathway P.

Because the implant has been diametrically compacted and collapsed upon itself it generally defines void spaces, or openings in the spaces between the diametrically compacted portions of the implant 610 (represented schematically by wavy lines in FIG. 9). As the blood flows along the blood pathway P, into these void spaces, air entrapped or entrained in the implant 610 in these void spaces is forced from the device. This mechanism may include the air being absorbed by the blood (e.g., hemoglobin) and/or the air being forced via fluidic pressure from the void spaces and out the proximal end of the implant 610. In different terms, when sufficient back pressure is applied (e.g., by mean arterial pressure, or MAP), the blood forces its way through any folds, creases, or gaps present in the implantable device 610 or that are present between the implantable device and retention sleeve 630. The pressurized blood then passes “through” the gaps past the closed valve 200 and out from the treatment system 100.

It can be particularly helpful that the valve 200 is closed around the outer perimeter of the implantable device 610 and any associated retention mechanism, as the treatment medium is less apt to simply pass around the implantable device 610 and any retention mechanism, but is instead forced to pass through the gaps, folds, creases and spaces in which air may be entrapped. As shown, the implant 610 and the retention sleeve 630 project proximally from the valve 200 a projection distance D. In some examples, the projection distance D is greater than 0 cm and less than 1 cm. In some examples, the projection distance D is approximately 1 cm. In some examples, the projection distance D is at least 1 cm. In particular, it has been surprisingly found that a projection distance of greater than 0 cm (e.g., approximately 1 cm) is particularly effective for reducing entrained air in the implant 610.

In some embodiments, the retention sleeve 630 and/or the implant 610 includes a visual insertion marker 612 (FIG. 1) (e.g., an implant visual insertion marker 612 and/or a retention sleeve visual insertion marker 612) for a user to confirm a proper projection distance D has been achieved. For example, the visual insertion marker 612 may be a visual band (e.g., ink, different material, or other marker) associated with the retention sleeve 630, the implant 610, or both. In some examples, the retention sleeve 630 is transparent or translucent, such that the visual insertion maker 612 is associated with the implant 610 and visible through the retention sleeve 630. The visual insertion marker 612 may include a visible, circumferential band, symbol, text, or other indicator to guide a user as to the proper depth of insertion for the endoluminal device in the valve 200 and the corresponding projection distance D.

In various examples, once the implantable device 610 has been treated as desired (e.g., flushed of air, wetted out, or otherwise treated), the treatment system 100 may be introduced into the body of a patient and delivery of the implantable device 610 may proceed in a desired manner (e.g., expanded and deployed from the delivery catheter 620).

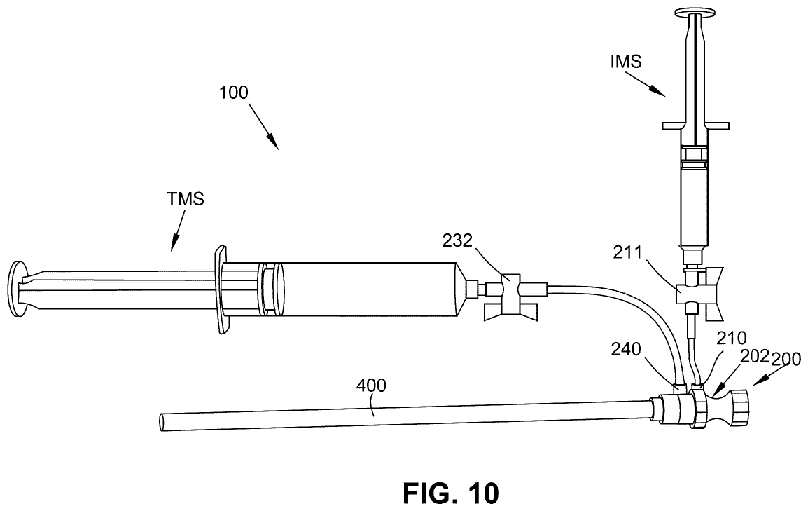

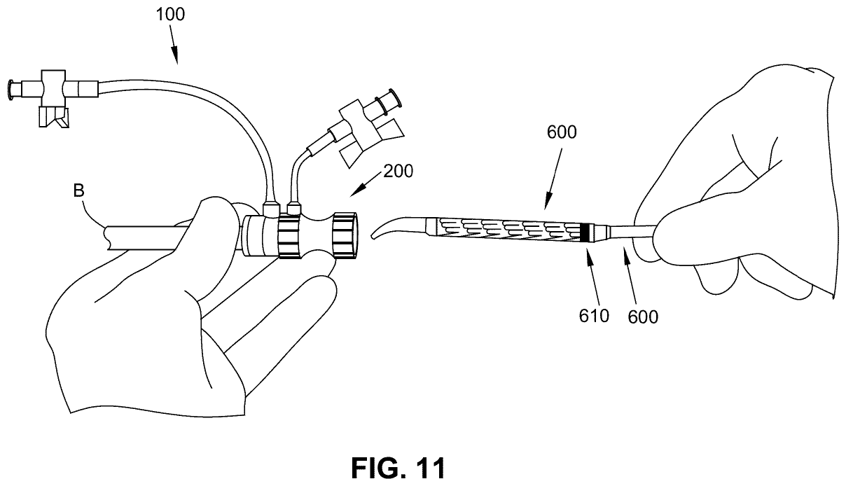

FIGS. 11 to 13 illustrate an example treatment sequence for an endoluminal device using the treatment system of FIG. 1, according to some embodiments. As shown in FIG. 10, an inflation media source (IMS) and a flush media source (TMS) are coupled to the fill port 210 and the flush port 240 of the valve 200, respectively. The inflation media source (IMS) may be a syringe filled with inflation media (e.g., saline) for pressurizing and depressurizing the valve 200 to open and close the valve 200. The flush media source (TMS) is optionally a syringe filled flush media (e.g., saline).

FIG. 11 shows the delivery sheath 400 inserted into the body B of the patient for accessing a blood vessel (not shown). FIG. 11 also shows endoluminal device 600 (e.g., in the form of transcatheter delivery system including a catheter and implantable device maintained at a compacted, delivery diameter or state by the delivery catheter) just prior to being introduced into the valve 200 of the treatment system 100.

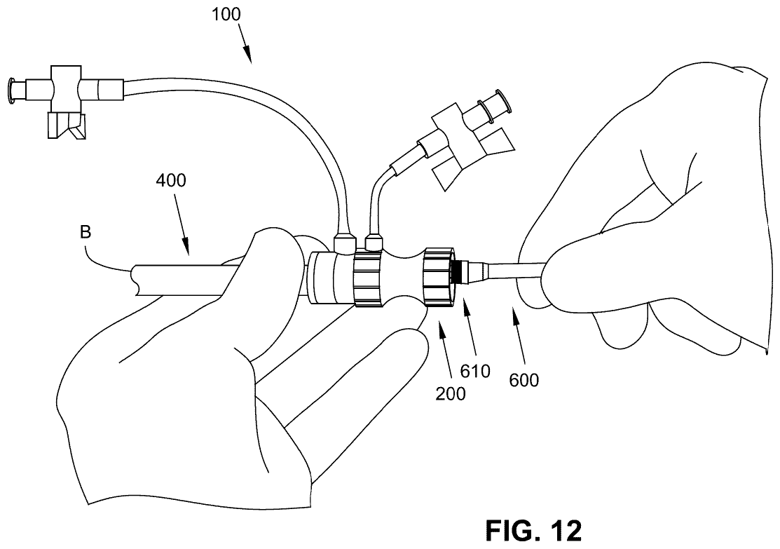

FIG. 12 shows the delivery sheath 400 inserted into the body B of the patient for accessing a blood vessel (not shown). The endoluminal device 600 introduced into the valve 200 with the implantable device 610 partially extending from the valve 200, such that a portion of the implantable device 610 is in the delivery sheath 400 and a portion projects from the valve 200. The valve 200 is pressurized using the inflation media source (IMS) (FIG. 12) to transition the valve 200 to a pressurized, closed state such that the valve 200 is closed over the implantable device 610.

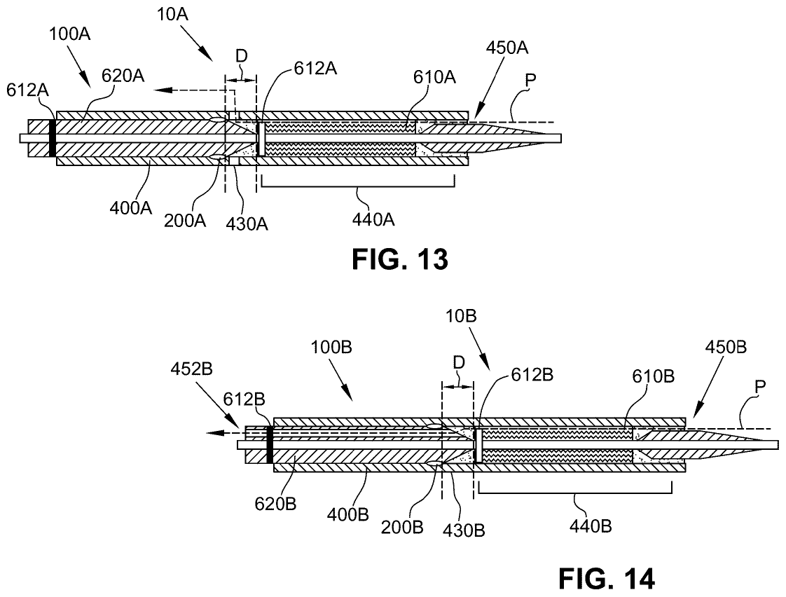

FIG. 13 is a schematic, sectional view of another endoluminal device delivery system 10A, according to some embodiments. The system 10A includes a treatment system 100A including a seal 200A, a delivery sheath 400A and an endoluminal device 600A (e.g., in the form of a transcatheter delivery system including a delivery catheter 620A and an implantable device 610A maintained at a compacted, delivery diameter or state on the delivery catheter 620A). The delivery sheath 400A and/or the endoluminal device 600A (e.g., delivery catheter 620A) includes the seal 200A for providing a hemostasis seal between the delivery sheath 400A and the endoluminal device 600A. The seal 200A may be an o-ring, for example, or other mechanism (e.g., an actuatable, inflatable seal). In other embodiments, the seal 200A is simply a sufficiently tight fit between the delivery catheter 620A and the delivery sheath 400A to provide sufficient resistance to blood flow to cause blood to flow along blood pathway P. As indicated, the delivery sheath 400A also includes one or more vents 430A and a distal section 440A terminating in a distal end 450A.

As shown schematically in FIG. 13, the implant 610A optionally includes an implant visual insertion marker 612A corresponding to the projection distance D (e.g., a radiographic material). Additionally or alternatively, the delivery catheter 620A optionally includes a delivery catheter visual insertion marker 612A corresponding to the projection distance D. In use, the visual insertion markers may be utilized to ensure a proper insertion distance D is achieved.

Generally, the distal section 440A is configured to be inserted into a body of a patient (not shown), and more specifically into the vasculature (arterial vasculature) of a patient. The implant 610A (which may be an endoprosthesis or any of the implants previously described) is generally constrained by the delivery sheath 400A in a compacted state, thereby defining folds, void spaces, or other spaces that may contain air in a similar manner to that previously described. A blood pathway P is defined from the open distal end 450A of the delivery sheath 400, between the delivery sheath 400A and the implant 610A, through the void spaces, or openings, containing air, and then out from the one or more vents 430A in the delivery sheath 400A. In use, the distal section 440A may be sufficiently long (and flexible) to be inserted into the vasculature while projecting at least somewhat from the body, with the one or more vents 430A being at an external position to the body. In this manner, the pressure differential between the interior blood pressure (e.g., mean arterial pressure) and ambient external pressure drives blood along the blood pathway P. In a similar manner to that previously described, the air contained in the implant 610A and/or between the implant 610A and the delivery sheath 400A may be substantially reduced or removed.

FIG. 14 is a schematic, sectional view of yet another endoluminal device delivery system 10B, according to some embodiments. The system 10B includes a treatment system 100B including a seal 200B, a delivery sheath 400B and an endoluminal device 600B (e.g., in the form of a transcatheter delivery system including a delivery catheter 620B and an implantable device 610B maintained at a compacted, delivery diameter or state on the delivery catheter 620B). The delivery sheath 400B and/or the endoluminal device 600B (e.g., delivery catheter 620B) includes the seal 200B for providing a hemostasis seal between the delivery sheath 400B and the endoluminal device 600B. The seal 200B may be an o-ring, for example, or other mechanism (e.g., an actuatable, inflatable seal). In other embodiments, the seal 200B is simply provided by a sufficiently tight fit between the delivery catheter 620B and the delivery sheath 400B to provide sufficient resistance to blood flow to cause blood to flow along blood pathway P.

As indicated, the delivery catheter 620B and/or the sheath 400B includes one or more vents 430B. The one or more vents 430B may be in the form of a vent tube, lumen, or snorkel, providing a fluid conduit from in front of the seal 200B to a proximal opening 452B located toward the proximal end of the treatment system 100B. This proximal opening 452B should be at a position on the treatment system 100B that can be located external to the body of the user, thereby providing the aforementioned pressure differential between ambient pressure and blood pressure, forcing blood flow along the blood pathway P. Similarly, the delivery sheath 400B includes a distal section 440B on a distal side of the seal 200B and terminating at a distal end 450B.

As shown schematically in FIG. 14, the implant 610B optionally includes an implant visual insertion marker 612B corresponding to the projection distance D (e.g., a radiographic material). Additionally or alternatively, the delivery catheter 620B optionally includes a delivery catheter visual insertion marker 612B corresponding to the projection distance D. In use, the visual insertion markers may be utilized to ensure a proper insertion distance D is achieved.

Generally, the distal section 440B is configured to be inserted into a body of a patient (not shown), and more specifically into the vasculature (arterial vasculature) of a patient. The implant 610B (which may be an endoprosthesis or any of the implants previously described) is generally constrained by the delivery sheath 400B in a compacted state, thereby defining folds, void spaces, or other spaces that may contain air in a similar manner to that previously described. A blood pathway P is defined from the open distal end 450B of the delivery sheath 400, between the delivery sheath 400B and the implant 610B, through the void spaces, or openings, containing air, and then out from the one or more vents 430B in the delivery catheter 620B and/or delivery sheath 400B. In use, the distal section 440B may be sufficiently long (and flexible) to be inserted into the vasculature, and the overall treatment system 100 being configured to be inserted into the body of a patient (not shown) with the one or more vents 430B at an external position to the body. Again, in this manner, the pressure differential between the interior blood pressure (e.g., mean arterial pressure) and ambient external pressure drives blood along the blood pathway P. In a similar manner to that previously described, the air contained in the implant 610B and/or between the implant 610B and the delivery sheath 400B may be substantially reduced or removed.

As described in further detail, some methods of pre-treating an endoluminal device for introduction into a body of a patient includes using a back-bleeding technique. Some methods of pre-treating an endoluminal device by back-bleeding include positioning a treatment system in a body lumen of a patient such that a distal portion of the treatment system is exposed to blood at a blood pressure and inserting the endoluminal device including an implant in a diametrically compacted, collapsed state and a delivery catheter into the treatment system at a pre-selected projection distance. The treatment system includes a delivery sheath and a seal (e.g., hemostatic seal provided by a valve, o-ring, seal or other mechanism) between the endoluminal device and the treatment system. Blood is permitted to flow along a blood pathway defined between the distal portion of the treatment system and a proximal portion of the treatment system (e.g., using a pressure differential between blood pressure and ambient or environmental pressure). The blood pathway extends through a first end of the implant to the second end of the implant and into one or more void spaces defined by the implant such that a volume of entrained air in the one or more void spaces is reduced.

The volume of the entrained air may be reduced to 10 μL or less, 5 μL or less, 2 μL or less or other value as desired. A least one of the implant, the delivery catheter, and a retention sleeve of the endoluminal device may include a visual insertion marking on an outer surface thereof that corresponds to the pre-selected projection distance, wherein the method includes inserting the endoluminal device into the treatment system to the pre-selected projection distance using the visual insertion marking. Some embodiments include using the visual insertion marking with the unaided eye. Ins some examples, the back-bleeding method includes observing 2 to 3 drips of blood from the blood pathway. In some examples, the method includes back-bleeding the implant for at least 5 seconds, 10 seconds, 15, seconds, 30 seconds, 1 minute, or 5 minutes, for example. In some methods, the blood pressure corresponds to mean arterial pressure (MAP), such as 67 mmHg, or more than 67 mmHg.

### Air Release Test Method

It should be understood that although certain methods and equipment are described below, other methods or equipment determined suitable by one of ordinary skill in the art may be alternatively utilized.

### Purpose

The purpose of the Air Release Testing (ART) is to quantify the air that is released during the deployment of endoluminal devices using no back-bleed and back-bleed methods.

### Equipment

2.5 ml, 10 ml, and 60 ml syringes
Hamilton 50 μL, Model 1705 TLLX, Instrument Syringe
Air Bubble Characterization Fixture
0.035 in Guidewire
Pressure Fluid Chamber
Blood Analog
Water bath for air transfer

### Test Setup

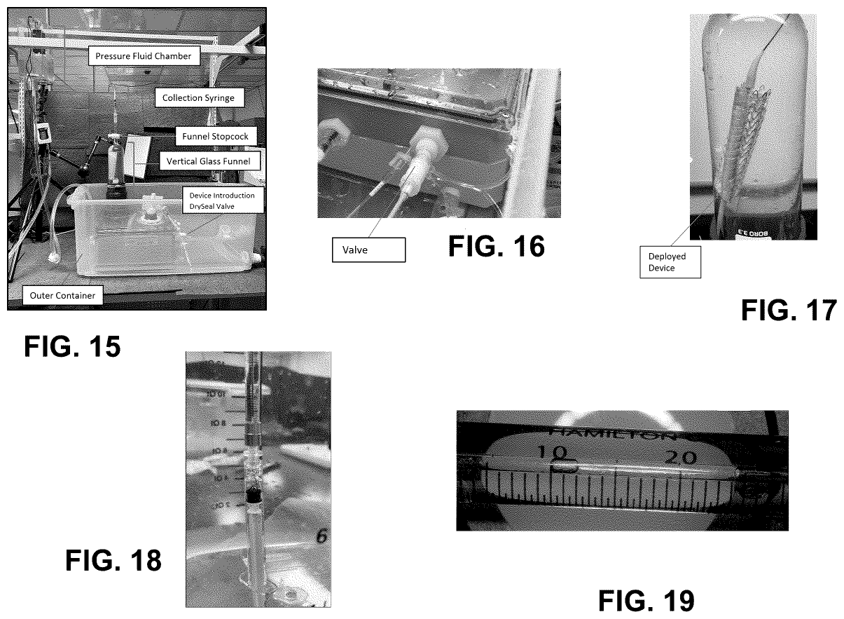

FIG. 15 shows an Air Bubble Characterization (ABC) fixture placed in an Outer Container with a Vertical Glass Funnel and a Collection Syringe attached towards the top of the fixture. This system is connected via tubing to a Pressure Fluid Chamber on the left which is at the appropriate height to simulate Blood Pressure (e.g., mean aortic pressure). The Test Set Up includes the following steps:

Step 1.1 Setting up the Air Bubble Characterization (ABC) fixture by placing it in an outer container and attaching the vertical glass funnel at the top of the fixture. Ensure the pressure fluid chamber attached to the fixture is set to a height such that the manometer reads the appropriate Blood Pressure (67 mmHg) for the test.

Step 1.2. Placing a pump in a bucket of blood analog and ensure the blood analog can be pumped through the tube to the Pressure fluid chamber.

Step 1.3. Ensuring tubing is connected between the Pressure fluid chamber and the ABC fixture.

Step 1.4. Inflating all Gore DrySeal valves connected to the fixture with 2.5 ml of water.

Step 1.5. Once the ABC fixture is filled with blood analog, closing and securing the plastic lid of the ABC fixture and continuing to fill the rest of the fixture with blood analog.

Step 1.6. Ensuring the funnel stopcock on the vertical glass funnel is open.

Step 1.7. Letting the system stay at a steady-state flow for a few hours or until air bubbles in the vertical glass funnel are no longer seen.

### Catheter Flush

The Catheter Flush includes: Step 2.1. Flushing the catheter of each device per instructions for use (IFU) flushing procedures. Step 2.2. Documenting the catheter flushing procedure that was followed (e.g., IFU number and revision). Step 2.3. Proceeding to desired device flushing step below.

### Device Flushing

The device is pre-treated (e.g., flushed) or not as part of the test protocol. One pre-treatment method includes use of a GORE® DrySeal Sheath valve. FIG. 16 shows an endoluminal device (Test Article) inserted into a pre-treatment device (GORE® DrySeal Sheath valve with approximately 1 cm exposed from the back side of the valve such that fluid can weep from the exposed portion of the endoluminal device −2 to 3 visible drips of fluid weep from the exposed portion of the endoluminal device). The device flushing method including use of the GORE® DrySeal Sheath Valve includes the steps of:

3.1. Ensuring the fluid chamber on tester is filled with blood analog to a level corresponding to the appropriate blood pressure (e.g., mean aortic pressure, or MAP) within a tolerance of +/−5 mmHg. Recording the reading of the pressure at the Valve flush port from the manometer.

3.2a No Back-Bleed. Inserting Test Article fully into the GORE® DrySeal Sheath valve. Proceeding to Device Insertion step below.

3.2b Back-Bleed. Inserting the Test Article into the Valve leaving approximately 1 cm of the component to be flushed (crushed stent graft) exposed. Wait until fluid (blood analog) begins to weep from the exposed portion of the component. Once fluid consistently weeps from the exposed portion of the component (2-3 drips of fluid), back-bleeding is complete. Ensure the projection distance D is about 1 cm with the GORE® DrySeal Sheath valve. Proceed to Device Insertion step below.

### Device Insertion and Deployment

FIG. 17 shows deployment of a device following pre-treatment in a Vertical Glass Funnel. The Device Insertion and Deployment includes the steps of:

4.1. Ensuring entire air bubble characterization fixture system is purged of air by purging any existing air out of the measurement syringe and stopcocks.

4.2. Ensuring the funnel stopcock on the Vertical Glass Funnel is open. Inserting an appropriate guidewire(s) through the Valve.

4.3. Advancing the test article over the guidewire(s) through the Valve and into the ABC fixture until the entire flushed component (crushed device) can be visualized in the Vertical Glass Funnel.

4.4. Completing device deployment procedure per the applicable sections of the associated Test Article documentation.

### Air Collection and Measurement

FIG. 18 shows a glass syringe attached to a 2.5 mL Collection Syringe via a luer coupler. FIG. 19 shows Image of air volume in the glass syringe. The assembly is inverted such that the glass syringe is at the top for air transfer. Upon deployment of the flushed (or not flushed) device, air bubbles are allowed to float up and collect below the Collection Syringe. The Air Collection and Measurement protocol includes the steps of:

5.1. Rotating the Funnel Stopcock and allow any trapped air bubbles along the edges to float up and collect below the Collection Syringe.

5.2. Using the plunger to draw the air bubbles into the Collection Syringe.

5.3. Disconnecting the Collection Syringe and take it to the water bath for air transfer.

5.4. Submerging the syringe of collected device air volume into the water bath and connect it to a luer coupler.

5.5. Depressing the 2.5 mL syringe slightly, without releasing any of the collected air volume, to ensure no air remains in the coupler.

5.6. Attaching a 50 μl glass syringe to the luer coupler.

5.7. Inverting this syringe assembly underwater such that the glass syringe is at the top.

5.8. Tapping the assembly until all bubbles break free from the plunger of the 2.5 mL Collection Syringe and collect the air volume at the luer coupler.

5.9. Using the glass plunger to pull all the air volume into the 50 μl glass syringe.

5.10. Recording total volume of air in the glass syringe.

### Test Method Discussion

A blood pressure (e.g., mean aortic pressure) of 67 mmHg may be used for testing as a clinically relevant and potential worst case (e.g., low pressure) scenario for air release quantification testing. In order to represent clinical settings, a pressurized system is used to simulate physiological aortic pressures. As applicable, the GORE® DrySeal Valve may be used per the Instruction for Use for that device. The fluid column is filled and attached to an extendable tripod and set to a height of approximately 67 mmHg (e.g., starting from the GORE® DrySeal Valve into which the devices would be introduced). A vertical glass funnel is used to allow devices to deploy and allow the air to float upwards for collection with a syringe.

Suitable blood analog is prepared such that the viscosity of blood analog at 25° C. matches the viscosity of blood at 37° C. to mimic the viscosity of blood in vivo. Testing of the blood analog may be completed at ambient temperature. The geometry of the deployment model or the temperature of the blood analog should not affect the amount of air that is released during the device deployment. Therefore, any representative deployment model and blood analog at ambient temperature are acceptable. Viscosity testing should be performed to ensure the solution matches hematocrit blood.

### Data Analysis

At the completion of deployment, total volume of released air should be reported for each device size and flush method group. The mean, standard deviation, maximum, and minimum values can be reported for each flush method group per device size. The volume of released air can be quantified and compared between the flush method groups.

### EXAMPLES

### Example 1

### Test Articles

GORE® TAG® Thoracic Branch Endoprosthesis Aortic Component (“TBE Aortic Component”), available from W. L. Gore & Associates, Inc., headquartered in Newark, DE.

GORE® TAG® Thoracic Branch Endoprosthesis Side Branch (SB) Component (“TBE Side Branch”), available from W. L. Gore & Associates, Inc., headquartered in Newark, DE.

GORE® TAGR Conformable Thoracic Stent Graft with ACTIVE CONTROL System (“CMDS”), available from W. L. Gore & Associates, Inc., headquartered in Newark, DE.

Table 1 lists the Test Articles by product and sample size for each.

TABLE 1
Test Articles and Sample Size

| | | Flushing | Sample |
|---|---|---|---|
| Product | Size | Technique | Size |
| TBE Aortic | 45 mm × 45 mm × | Back-bleed | 10 |
| Component | 20 cm, 8 mm portal | | |
| | 45 mm × 45 mm × | Back-bleed | 10 |
| | 20 cm, 12 mm portal | | |
| TBE Side | 12 mm × 20 mm × 6 cm | Back-bleed | 10 |
| Branch | 12 mm × 20 mm × 6 cm | No Back-bleed | 10 |
| CMDS | 45 mm × 45 mm × 20 cm | Back-bleed | 10 |
| | 45 mm × 45 mm × 20 cm | No Back-bleed | 10 |
| CMDS | 31 mm × 26 mm × 10 cm | Back-bleed | 10 |
| | 31 mm × 26 mm × 10 cm | No Back-bleed | 10 |

All samples were sterilized, standard devices and assessed to verify no defects that would affect the attribute under evaluation.

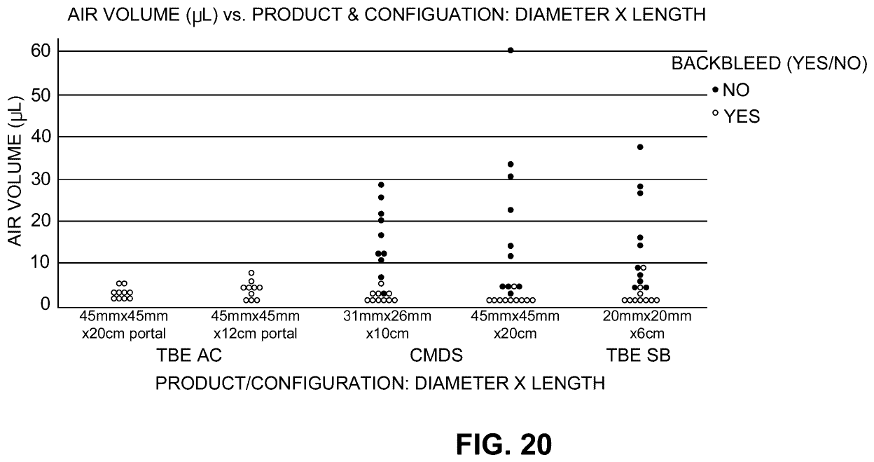

FIG. 20 graphically represents the air volume (μL) for the devices tested using the Air Release Test Method described above for the Test Articles identified in Table 1. The devices were tested using the Back-Bleed and No Back-Bleed flushing techniques, MAP of 67 mmHg, and the GORE® DrySeal Introducer Sheath valve was inflated with 2.5 ml of water. For all Back-Bleed data, the projection distance D was about 1 cm.

In order to assess the potential air in untreated devices that have not yet been passed through the GORE® DrySeal Introducer Sheath, testing was conducted without utilizing mean arterial pressure (MAP) or the DrySeal valve technology/back-bleeding. Without any such interaction (i.e., simply deploying samples of the Test Articles) generally showed the volume of air entrapped in devices could range from 100-1,020 μL.

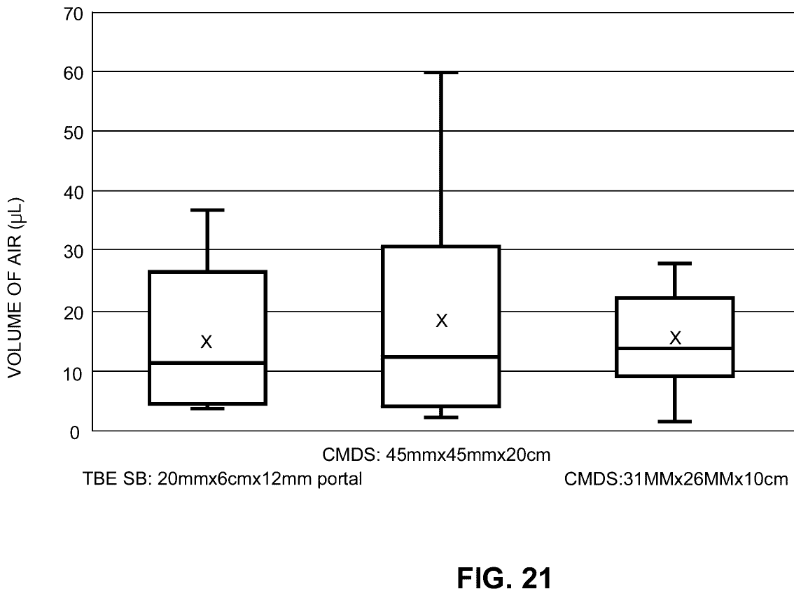

The No Back-Bleed testing was performed by incorporating a MAP of 67 mmHg, and using a GORE® DrySeal Sheath valve inflated with 2.5 mL of saline. The endoprosthesis was passed through the GORE® DrySeal Sheath valve without waiting for back-bleeding. In such instances, the total volume of air entrapped in devices was measured at an average of 15.983 μL (e.g., reduced from an average ranging between 100 and 1,020 μL). FIG. 21 presents data from that testing in graphical form. The No Back-Bleed testing resulted in a 95.0% reduction in the volume of air entrapped compared to units pulled directly from production (322 μL-15.983 μL)/322 μL.

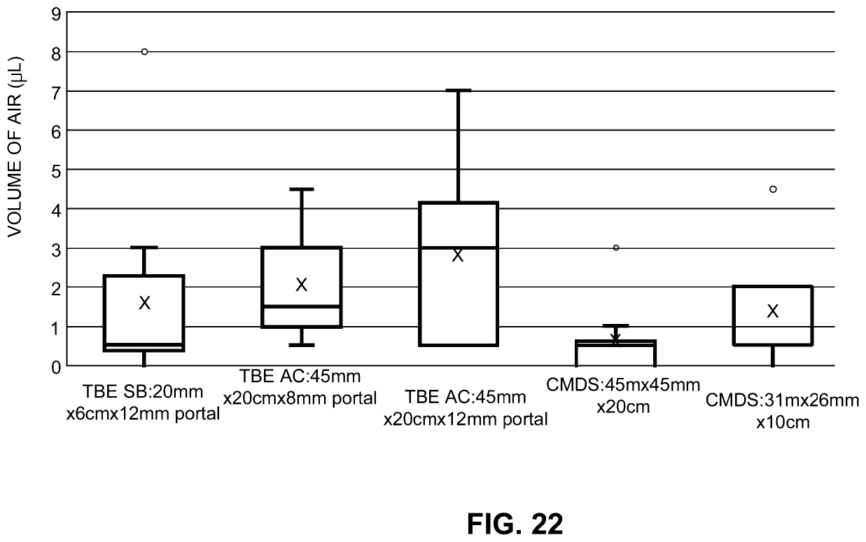

The Back-Bleed testing was performed by incorporating a MAP of 67 mmHg, and a GORE® DrySeal Introducer Sheath valve inflated with 2.5 mL of saline, and back-bleeding (dwell time sufficient to exhibit 2 to 3 drips of back-bleeding), the volume of air entrapped in devices was reduced to an average of 1.720 μL. This is a 99.5% reduction (322 μL-1.720 μL)/322 μL compared to units pulled from production, and an 89.2% reduction in the volume of air compared to using a MAP of 67 mmHg, a DrySeal Sheath valve inflated with 2.5 mL of saline, without backbleeding (15.983 μL-1.720 μL)/15.983 μL. FIG. 22 presents data from that testing in graphical form.

### Example 2

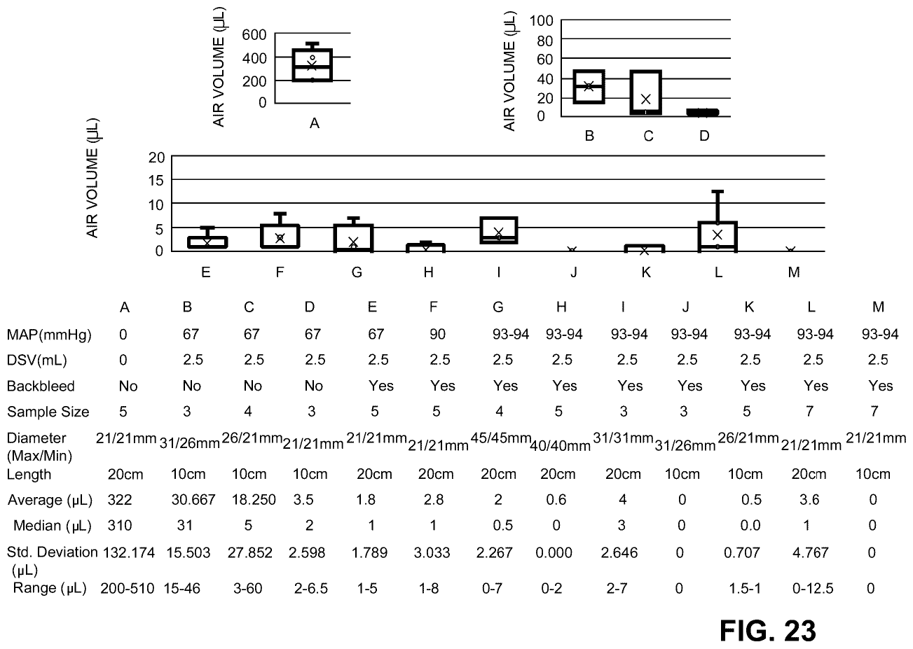

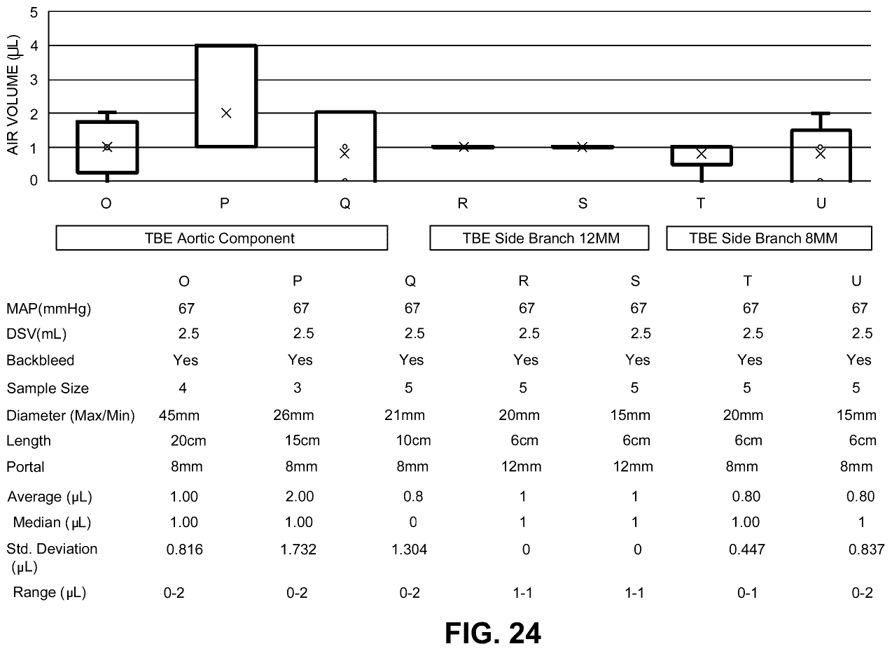

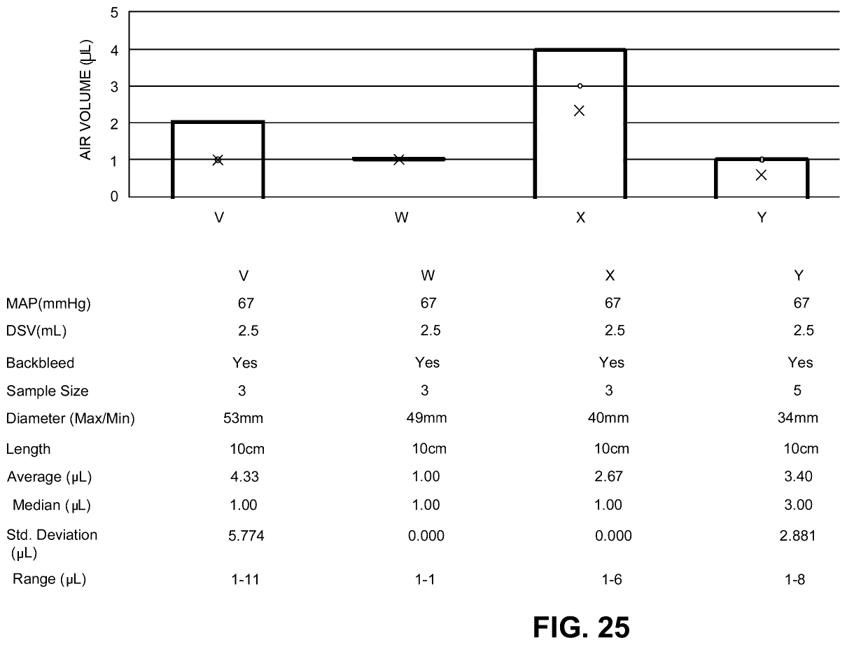

FIGS. 25 to 27 present additional data for various Test Articles tested according to the Air Release Test Method and Back-Bleed or No Back-Bleed testing using a blood analog. FIG. 23 represents data for various configurations of the Conformable GORE® TAG® Thoracic Endoprosthesis (“CTAG”), available from W. L. Gore & Associates, Inc., headquartered in Newark, DE. FIG. 26 represents data for various configurations of GORE® TAG® Thoracic Branch Endoprosthesis Aortic Component (“TBE Aortic Component”) (Data sets O, P, and Q) and GORE® TAG® Thoracic Branch Endoprosthesis Side Branch (SB) Component (“TBE Side Branch”) (Data sets R and S, and T and U), available from W. L. Gore & Associates, Inc., headquartered in Newark, DE. FIG. 27 represents data for various configurations for the GORE® Ascending Stent Graft (“ASG”), manufactured by W. L. Gore & Associates, Inc. The various variables and data are described in the foregoing Test Method and Example 1 sections. Notably, Example 2 includes some data at higher mean arterial pressures (MAPs) than conducted in the testing in Example 1. For reference, the data sets (such as Data Set A from FIG. 23) that include a MAP of “0” correspond to data taken without use of the GORE® DrySeal Introducer Sheath, or an “as manufactured” measurement without an insertion and exposure to MAP. For all Back-Bleed data, the projection distance D was about 1 cm.

FIG. 23 presents comparative data between Data Sets A, E, F, and L. As indicated the entirely untreated device (e.g., as manufactured) has an average starting air content of around 322 μL (Data Set A). Using a Back-Bleed method and Air Release Test Method at 67 mmHg MAP (Data Set E), 90 mmHg MAP (Data Set F), and 93-94 mmHg MAP (Data Set L) resulted in a drop to an average air content remaining of less than 5 μL, and less than 2 μL (1.8 μL) in the instance of 67 mmHg.

What is claimed is:

1. An endoluminal device delivery system comprising:
an endoluminal device including,
an implant having a first end and a second end, the implant being configured in a collapsed, delivery configuration, the implant having one or more void spaces defined by the collapsed implant and filled with air;
a delivery catheter including a body to which to the endoluminal device is mounted; and
a treatment system including,
a delivery sheath, and
a valve coupled to the delivery sheath, the valve being actuatable between a sealed and an unsealed configuration, the implant projecting proximally from the valve a projection distance of approximately 1 cm, the treatment system defining a blood pathway between the delivery sheath and the body of the catheter for blood to pass from the first end of the implant to the second end of the implant and into the one or more void spaces defined by the collapsed implant, the valve actuatable from an unsealed state in which blood bypasses the blood pathway to a sealed state in which blood is forced along the blood pathway.

2. The system of claim 1, wherein the implant projects proximally from the valve from 0.5 cm to 1.5 cm.

3. The system of claim 1, wherein the implant includes an implant visual insertion marker corresponding to the projection distance.

4. The system of claim 1, wherein the endoluminal device further includes a retention sleeve maintaining the implant in the collapsed, delivery configuration.

5. The system of claim 4, wherein the retention sleeve includes a sleeve visual insertion marker corresponding to the projection distance.

6. An endoluminal device delivery system comprising:
an endoluminal device including,
an implant having a first end and a second end, the implant being configured in a collapsed, delivery configuration, the implant having one or more void spaces defined by the collapsed implant and filled with air;
a delivery catheter including a body to which to the endoluminal device is mounted; and
a treatment system including,
a delivery sheath, and
a seal between the delivery catheter and the delivery sheath at a location proximal the implant corresponding to a projection distance between the seal and the implant, the delivery sheath having a distal end configured to be exposed to blood pressure and the treatment system having one or more vents configured to vent to ambient air pressure, the one or more vents having an opening located distal to the seal and proximal the implant such that the treatment system defines a blood pathway between the delivery sheath and the body of the catheter for blood to pass from the first end of the implant to the second end of the implant and out of the one or more vents, the blood being directed into the one or more void spaces defined by the collapsed implant.

7. The system of claim 6, wherein the one or more vents has an exit located distal to the seal.

8. The system of claim 7, wherein the one or more vents are formed in the delivery sheath.

9. The system of claim 6, wherein the one or more vents has an exit located proximal to the seal.

10. The system of claim 9, wherein the exit is located at the proximal end of the treatment system.

11. The system of claim 9, wherein the one or more vents are formed in the delivery catheter.

12. The system of claim 9, wherein the one or more vents are formed as a vent tube, lumen, or snorkel, providing a fluid conduit from distal to seal to a proximal opening located toward the proximal end of the treatment system.

13. The system of claim 6, wherein the implant includes an implant visual insertion marker corresponding to the projection distance.

14. The system of claim 13, wherein the implant visual insertion marker includes a radiographic material.

15. The system of claim 6, wherein the delivery catheter includes a delivery catheter visual insertion marker corresponding to the projection distance.

16. An endoluminal device delivery system having a pre-selected projection distance of an endoluminal device from a delivery sheath of the system, the system comprising:
an endoluminal device including,
an implant having an outer surface and being configured to transition from a diametrically compacted, delivery configuration to a diametrically expanded, deployed configuration, the implant having a proximal end, a distal end opposite to the proximal end, and length between the proximal and distal ends, and
a retention sleeve constraining the implant in the diametrically compacted, delivery configuration, wherein at least one of the implant and the retention sleeve includes a visual insertion marking on an outer surface thereof; and
a treatment system including a delivery sheath configured to introduce the endoluminal device into a body of a patient,
wherein the visual insertion marking corresponds to the pre-selected projection distance of the endoluminal device from the delivery sheath.

17. The system of claim 16 wherein the visual insertion marking is visible with the unaided eye.

18. The system of claim 16, wherein the visual insertion marking aligns with a proximal end of the treatment system at the pre-selected projection distance.

19. An endoluminal device delivery system having a pre-selected projection distance of an endoluminal device relative to a seal of a delivery sheath of the system, the system comprising:
an endoluminal device including,
an implant having a first end and a second end, the implant being configured in a collapsed, delivery configuration, the implant having one or more void spaces defined by the collapsed implant and filled with air;
a delivery catheter including a body to which to the implant is mounted; and
a treatment system including,
a delivery sheath, and
a seal between the delivery catheter and the delivery sheath at a location proximal the implant corresponding to a projection distance between the seal and the implant,
wherein at least one of the implant and the delivery catheter includes a visual insertion marking on an outer surface thereof corresponds to the pre-selected projection distance of the endoluminal device from the delivery sheath.

20. The system of claim 19, wherein the visual insertion marking is visible with the unaided eye.

21. The system of claim 19, wherein the visual insertion marking aligns with a proximal end of the treatment system at the pre-selected projection distance.

22. A method of pre-treating an endoluminal device by back-bleeding, the method comprising:
positioning a treatment system in a body lumen of a patient such that a distal portion of the treatment system is exposed to blood at a blood pressure;
inserting an endoluminal device including an implant in a diametrically compacted, collapsed state and a delivery catheter into the treatment system at a pre-selected projection distance, the treatment system including a delivery sheath and a seal between the endoluminal device and the treatment system;
permitting blood to flow along a blood pathway defined between the distal portion of the treatment system and a proximal portion of the treatment system, the blood pathway extending through a first end of the implant to the second end of the implant and into one or more void spaces defined by the implant such that a volume of entrained air in the one or more void spaces is reduced.

22. The method of claim 21, wherein the volume of the entrained air is reduced to 10 μL or less.

23. The method of claim 21, wherein the volume of the entrained air is reduced to 5 μL or less.

24. The method of claim 21, wherein the volume of the entrained air is reduced to 2 μL or less.

25. The method of claim 21, wherein at least one of the implant, the delivery catheter, and a retention sleeve of the endoluminal device includes a visual insertion marking on an outer surface thereof that corresponds to the pre-selected projection distance, and further wherein the method includes inserting the endoluminal device into the treatment system to the pre-selected projection distance using the visual insertion marking.

26. The method of claim 25, wherein the method includes using the visual insertion marking with the unaided eye.

27. The method of claim 21, wherein permitting blood to flow along the blood pathway includes observing 2 to 3 drips of blood from the blood pathway.
